(19) United States
(12) Patent Application Publication
(10) Pub. No.: US 2004/0229379 A1
(43) Pub. Date: Nov. 18, 2004

(54) Assay

(76) Inventors: Neil Benson, Sandwich (GB); David John Bull, Sandwich (GB); Peter John Bungay, Sandwich (GB); Andrew Lee Hopkins, Sandwich (GB)

Correspondence Address: Pharmacia Corporation, Global Patent Department, Mail Zone MC5, P. O. Box 1027, St. Louis, MO, 63141, US

(21) Appl. No.: 10/794,042

(22) Filed: Mar. 5, 2004

Related U.S. Application Data
(60) Provisional application No. 60/458,465, filed on Mar. 28, 2003.

(30) Foreign Application Priority Data
Mar. 5, 2003 (GB) 0305060.6

(51) Int. Cl. G01N 21/76; G01N 33/543

(52) U.S. Cl. 436/518

(57) ABSTRACT

An assay method for determining binding of a test compound to pyruvate dehydrogenase kinase (PDHK) at a given site comprising determining (a) the degree of polarisation of the light emitted by a PDHK complex formed by contacting in solution a known concentration of PDHK with a known concentration of a fluorescent compound known to bind to PDHK at that site, and (b) the degree of polarisation of the light emitted by a PDHK complex formed by contacting in solution the test compound, the same concentration of PDHK and the same concentration of the fluorescent compound, a lesser value of the degree of polarisation (b) than that of (a) being taken as indicative of binding of the test compound to that site.

### ASSAY

This application claims the benefit of U.S. Provisional application No. 60/458,465, filed Mar. 28, 2003, and United Kingdom application number 0305060.6 filed Mar. 5, 2003, which is incorporated by reference in its entirety.

This invention relates to an assay for identifying compounds that bind to and modulate the activity of pyruvate dehydrogenase kinase (PDHK) enzymes.

### BACKGROUND OF THE INVENTION

PDHK

Phosphorylation of pyruvate dehydrogenase (PDH) by pyruvate dehydrogenase kinase (PDHK; EC 2.7.1.99) (PDH+ATP—^{PDHK}→PDH-P+ADP) results in the inactivation of PDH and consequently a reduction in the rate of acetyl coenzyme A production from carbohydrate. Inhibitors of PDHK may thus act to promote the flux of carbohydrate through oxidative metabolism, and may help to improve its efficiency under adverse physiological conditions. Hence PDHK inhibitors may be useful as therapeutic agents in the treatment of diseases where oxidative metabolism of carbohydrate is compromised. Such conditions include peripheral vascular disease, where inhibition of PDHK may improve the performance of skeletal muscle and decrease fatigue such as occurs during intermittent claudication; and angina.

PDHK inhibitors have a potential role in cardioprotection, preventing myocardial infarction and improving recovery of ischaemic heart, thereby limiting tissue damage. Dichloroacetic acid (DCA) is a known PDHK inhibitor which is used experimentally as a cardioprotectant.

PDHK inhibitors may be useful it the treatment of diabetes by promoting metabolism of carbohydrate over metabolism of fat

Prior PDHK Assays

Spectrophotometric assays have been used to detect the rate of inactivation of the PDH complex in the presence of ATP. PDH complex is phosphorylated in the presence of ATP by endogenous PDHK. The phosphoryated PDH complex acts on CoA and pyruvate in the presence of NAD resulting in the production of NADH. NADH can be detected by spectrophotometric methods and thus acts as a reporter.

In an alternative assay PDHK is incubated with 33P ATP and an artificial protein substrate, resulting in incorporation of 33P into the protein. This radiolabel can be detected by scintillation counting. As an alternative, the protein substrate can be biotinylated and captured on a streptavidin filter; the bioinylated protein substrate is then detected by scintillation counting.

A disadvantage of the known assays is that it is difficult to screen large numbers of compounds rapidly. Furthermore, many compounds in the screening bank may absorb the waveleghths desired for use in spectrophotometric method and thus interfere with the readout of spectrophotometric assays.

PDHK Inhibitors

A series of compounds has been identified which bind to PDHK and inhibit its ability to phosphorylate and inactivate the PDH complex:

Aicher, T. D. et al., (2000) J. Med. Chem. 43, 236-249. Secondary amides of (R)-3,3,3-trifluoro-2-hydroxy-2-methylpropionic acid as inhibitors of pyruvate dehydrogenase kinase.

Aicher, T. D. et al., (1999) J. Med. Chem. 42, 2741-2746. (R)-3,3,3-trifluoro-2-hydroxy-2-methyl-propionamides are orally active inhibitors of pyruvate dehydrogenase kinase.

Jackson, J. C. et al., (1998) Biochem. J. 334, 703-711. Heterologously expressed inner lipoyl domain of dihydrolipoyl acetyltransferase inhibits ATP-dependent inactivation of pyruvate dehydrogenase complex.

Bebernitz, G. R. et al., (2000) J. Med Chem. 43, 2248-2257. Anilides of (R)-Trifluoro-2-hydroxy-2-methylpropionic Acid as Inhibitors of Pyruvate Dehydrogenase Kinase.

Aicher, T. D. et al., (2000) J. Med. Chem. 43, 236-249. Secondary amides of (R)-3,3,3-trifluoro-2-hydroxy-2-methylpropionic acid as inhibitors of pyruvate dehydrogenase kinase.

See also WO9944618 and WO9947508.

Within the classes of compounds disclosed in these publications are those characterized in that they contain the radical (I):

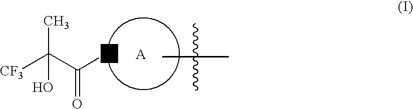

wherein ring A is mono- or bi-cyclic, this motif being assumed to be a determinant of PDHK binding capability.

Although there is no direct proof of the mechanism of inhibition of PDHK activity, it is possible that these compounds interact with the E2_{L2} binding domain on PDHK, which is important in mediating the interaction of PDHK with PDH.

### BRIEF DESCRIPTION OF THE INVENTION

This invention is based on the finding that compounds which bind to PDHK may be coupled to fluorescent compounds without loss of binding capacity and resultant inhibition of ATP-dependent inactivation of PDH, and that this capability allows the use of fluorescence polarisation as the read-out of an assay for determining the binding of test compounds to the enzyme. The complex of PDHK and fluorescent-coupled binder compound is approx. 100 times the mass of the free compound. Thus, if a polarized excitation beam is used to illuminate a mixture of bound and free compound, light emitted by compound bound to PDHK will remain more polarized than that emitted from the unbound compound due to the reduced rotational diffusion of the bound probe. As the measured polarization is proportional to the ratio of bound to free fluorescent-coupled binder, it can be used directly to construct binding isotherms and in competition assays to assess the affinity of compounds for PDHK isoenzymes.

### BRIEF DESCRIPTION OF THE DRAWINGS

FIG. 1 is a graph representing the binding of Compound 1 of the present invention to PDHK by titration against fixed concentrations of fluorescent binder A and PDHK2 and PDHK4.

### DETAILED DESCRIPTION OF THE INVENTION

Accordingly, the present invention provides an assay method for determining binding of a test compound to PDHK at a given site comprising determining (a) the degree of polarisation of the light emitted by a PDHK complex formed by contacting in solution a known concentration of PDHK with a known concentration of a fluorescent compound known to bind to PDHK at that site, and (b) the degree of polarisation of the light emitted by a PDHK complex formed by contacting in solution the test compound, the same concentration of PDHK and the same concentration of the fluorescent compound, a lesser value of the degree of polarisation (b) than that of (a) being taken as indicative of binding of the test compound to that site.

The assay may be used to assign an IC50 value representing the binding affinity to the test compound. For this purpose, the degree of polarisation (b) is determined for each of a range of concentrations of the test compound, in each case contacted with a fixed concentration of PDHK and fluorescent compound, and the resultant values are compared to estimate the concentration of test compound which reduces the degree of polarisation (a) by 50%.

PDHK inhibitor compounds of the class disclosed in the publications listed above may be coupled to fluorescent compounds to provide the fluorescent binder compounds used in the assay. For example, the fluorescent compound for use in the assay may be one which contains the radical (I):

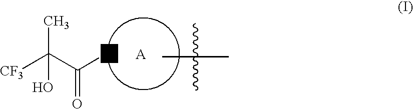

wherein ring A is mono or bicyclic. Details of such compounds are to be found in the literature cited above. In particular, the ring A may be a 2,5-dimethyl piperazinyl ring. It has been found also that the radical (II)

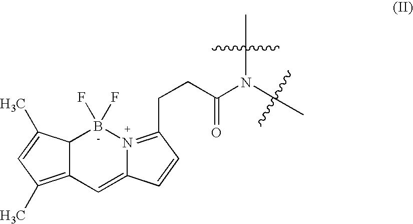

is a particularly suitable fluorescent motif for incorporation in the fluorescent binder compound for use in the assay. Such a radical may be introduced into a known binder compound containing an amino group R₂NH by reaction of the known fluorescent compound BODIPY FL of formula (IIA) to form an amido bond at the amino group in the binder compound R₂NH, thereby generating the desired fluorescent linked binder compound (IIB):

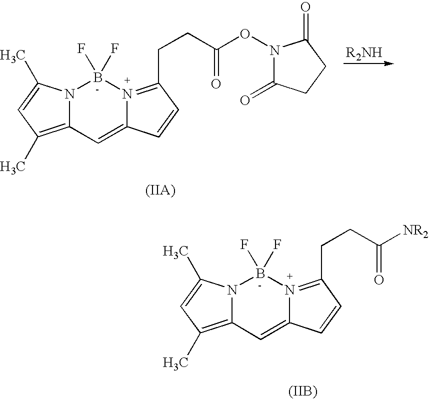

The presently preferred fluorescent binder compound for use in the assay is one having the structure (III).

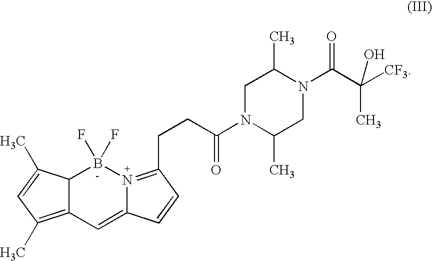

The most preferred stereoisomer of compound (III) is that of structure (IIIA)

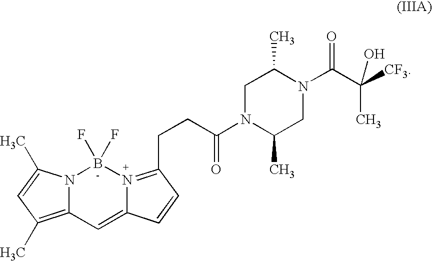

An example of another fluorescent binder structure is compound (IV)

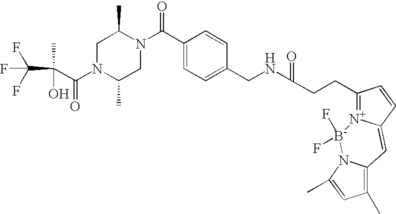

but it has been found to have a lower binding affinity for PDHK than (III/IIIA). In general fluorescent binder compounds which have higher binding affinities for the enzyme will be more preferred than those of lower affinities, when the assay is being used to screen for test compounds of high binding affinity. In this connection, the invention includes a method of screening a plurality of test compounds to identify any which bind to PDHK comprising subjecting the test compounds to the assay of the invention.

The following Example illustrates embodiments and principles of the invention.

### EXAMPLE

Methods

Expression and Purification of Human PDHK Isoforms

Recombinant PDHK-2 protein was generated by infection of Hi5 insect cells with a baculovirus encoding an N-terminally his-tagged version of PDHK-2 (amino acids 10-407). Cells were infected at a MOI of 10 for 48 h. Cell pellets were harvested and extracted in 50 mM Tris (pH 8.0), 150 mM NaCl, 5 mM mercaptoethanol, 20 mM imidazole (pH 8.0) and standard protease inhibitors. Soluble protein was purified by affinity chromatography on a Ni-chelate column. PDHK-2 was eluted from the Ni-column using a 40-500 mM gradient of imidazole (pH 8.0). The resulting protein was >95% pure.

Recombinant PDHK-4 protein was generated by expression in BL21pLysS bacterial cells. Full length PDHK-4 amplified from human skeletal muscle cDNA library (Clontech) was cloned into an N-terminally his-tagged pET vector (Novagen) and transformed into BL21 cells (BL21 plys5, Novagen). Cultures were allowed to reach an OD₆₀₀ of 0.6 before induction of PDHK expression by the addition of 1 mM IPTG. The cultures were incubated for a further 5 h at 30° C. with 220 rpm shaking. Bacterial cell pellets were harvested and extracted as described above. PDHK-4 was purified by a batch method utilising Ni-NTA agarose beads (Quiagen). The PDHK-4 eluted by addition of 500 mM imidazole was ˜95% pure.

Synthesis of Fluorescent-coupled PDHK Binder.

To a solution of 10 mg (0.023 mmol) of the known PDHK binder compound (IV)

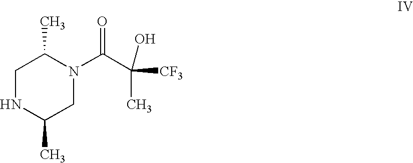

in 0.5 ml dimethylformamide was added 7 mg of the known fluorophore BODIPY FL (0.026 mmol; 1.1 eq) and the reaction mixture left stirring at room temperature. The solution changed from luminous yellow to dark red on initial addition of the fluorophore. The reaction mixture was partitioned between 10 ml each of ethyl acetate and water, and the organic phase washed with water, dried (magnesium sulphate) and concentrated under reduced pressure to yield a bright red residue. Flash chromatography eluting in toluene:ethyl acetate (4:1-1:1) yielded 10 mg product, which was further purified by dissolving in ether and treatment with 1 eq ethereal hydrochloric acid. Red solid precipitated which was filtered and dried under vacuum. Characterisation as structure (IIA)—see above—(“fluorescent binder A”) was by ¹H NMR and mass spectroscopy.

PDHK Solution

Stock solutions of PDHK (human isoforms 2 and 4) were stored at −80° C. (0.4-1.2 mg/ml). Aliquots were diluted in assay buffer to 4 times the required concentration. For competition assays, the final concentration required is that which gave approx. 50% maximal binding of the fluorescent-coupled binder and was approximately 10 nM in the case of PDHK2 and 60 nM for PDHK4. The exact concentrations required were determined using the saturation binding assay procedure (see below).

Fluorescent Binder A Solution

A 10 mM stock solution of fluorescent binder A was prepared in DMSO and aliquots stored at −20° C. 20 μl of this stock solution was added to 4.98 ml assay buffer to give a 40 μM solution. This was further diluted to 40 nM in assay buffer.

Binding Assay

The assay buffer consisted of 20 mM potassium phosphate, pH 7.8, containing 1 mM CHAPS. A 10 mM stock solution of fluorescent binder A was prepared in DMSO and further diluted to 40 nM in assay buffer (final DMSO concentration 0.004%). PDHK2 was diluted over the concentration range 2 μM to 0.5 nM and PDHK4 from 8 μM to 2 nM. To initiate binding reactions, 25 μl PDHK, 25 μl fluorescent binder 1 and 50 μl assay buffer were added to the wells of black microtitre plates and incubated at 25° C. for 60 min. Polarization values were then determined in a BMG PolarStar plate reader.

Stocks of test compounds were initially dissolved in 100% DMSO to 10 mM and were further diluted in water to 4 times the final required concentration. Typically, the highest concentration of compound used was 120 μM (corresponding to 30 μM in the assay), so that the DMSO concentration added was 1.2% (v/v). This concentration of DMSO in water was used as diluent for serial dilutions of compounds.

Assay Assembly for Competition Assay

The PDHK binding assay used a final volume of 100 μl and was performed in flat-bottom 96-well black microtitre plates (Costar) using duplicate determinations for each concentration of test compound. The incubation mixture consisted of 25 μl test compound in 1.2% DMSO, or 1.2% DMSO for controls; 25 μl 40 nM fluorescent binder 1; 25 μl assay buffer and, to initiate the binding reaction, 25 μl PDHK at 4 times the required concentration.

A set of at least three control wells for determination of minimum binding polarization values were also included in which PDHK is replaced by assay buffer. Plates were shaken, and, after incubation for 60 min, read in the Polarstar Galaxy, set up in polarization mode using excitation and emission wavelengths of 485 and 520 nm respectively.

The plate reading procedure consisted firstly of gain setting, for which one of the control wells from which PDHK was omitted is used. The gain is adjusted so as to achieve 90% of maximum reading and a K factor of 1.0±0.05. Polarization was measured subsequently for the entire plate after orbital shaking for 5 s.

Assay Assembly for Saturation Binding Assay (Kd Determination)

Serial dilutions of PDHK were prepared over the final concentration range 500-0.49 nM for PDHK2 and 2000 to 1.95 nM for PDHK4 using halving dilutions. Duplicate reactions were set up in black 96-well plates for each concentration of PDHK. Reaction mixtures consisted of 25 μl PDHK, 50 μl assay buffer and 25 μl 40 nM fluorescent binder 1. At least 3 wells were included in which assay buffer replaced enzyme (minimum binding). Plates were shaken, and, after incubation for 60 min, read in the Polarstar Galaxy, as described above for the competition assay.

Kd Determination

Data was captured using the BMG instrument control software linked to Microsoft Excel and polarization values cut and pasted as desired into an Excel spreadsheet. Data was then pasted into Grafit and fitted to a binding isotherm using an explicit function which takes into account depletion of free ligand due to binding. Kd values obtained were used in the calculation of Ki values for inhibitors of fluorescent binder 1 binding (see below).

rf+((rb−rf)*((Kd+F+L)−(sqrt(sqr(Kd+F+L)−4*F*L)))/(2*F))

rb=bound polarization

rf=free fluor polarization

F=added fluor conc.

L=ligand conc. added

Competition Data: Determination of Ki

Data was captured using the BMG instrument control software linked to Microsoft Excel and polarization values cut and pasted as desired into an Excel spreadsheet. Values for polarization were then pasted into GraphPad Prism against the appropriate concentration of test compound. Data were fitted to a Sigmoidal dose-response curve (variable slope) with the top and bottom constrained according to the control (absence of test compound=maximum binding) and minimum (no enzyme) polarization values respectively. The IC₅₀ value returned is then used to calculate a Ki value using the exact correction of Munson and Rodbard (Munson & Rodbard, 1988; 1989-1990) according to the equation:

Ki=IC₅₀/(1+L(y₀+2)/(2*Kd(y₀+1))+y₀))−Kd(y₀/(y₀+2))

where:

L=concentration of ligand added (fluorophore UK-421,090);

Kd=dissociation constant for PDHK:UK-421,090 interaction;

y₀=initial bound/free ratio for UK-421,090.

y₀ was calculated from the data obtained from control, maximum and minimum binding determinations from:

y₀=(control−min.)/(max.−min.))/1−(control−min.)/(max.−min.)

Results

1. Binding of Fluorescent Binder A to Various [PDHK]

In order to investigate the affinity of binding of fluorescent binder A to recombinant PDHK enzymes, serial dilutions of PDHK2 and PDHK4 were prepared and aliquots incubated with a constant concentration (10 nM) of fluorescent binder 1. The change in fluorescence polarization value obtained was plotted against log [PDHK] added, and data fitted to the equation given in the methods section. The results for binding to fluorescent binder A showed that PDHK2 bound with high affinity (Kd=4.3 nM) and that PDHK4 bound with lower affinity (Kd=61 nM).

2. Competition Assay Based on UK-421,090 Binding

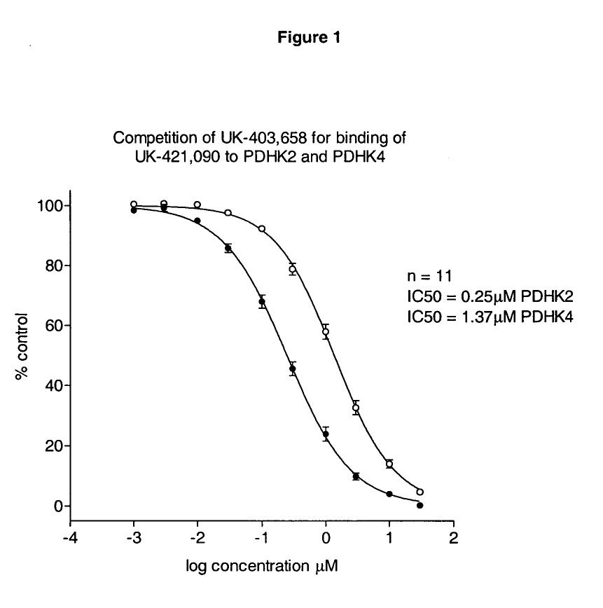

A range of compounds were screened for binding to PDHK by titration against fixed concentrations of fluorescent binder A and PDHK2 and PDHK4. Some were found to compete with binding of fluorescent binder A in a concentration-dependent manner as exemplified by Compound 1 in FIG. 1.

The results for those compounds identified as binding (Table 1) show that within the series of compounds a range of potencies exist in competition for the binding of fluorescent binder A to PDHK enzymes. Furthermore, the assay was able to differentiate compounds which possessed similar affinity for PDHK2 and 4 (e.g. compound 9) from those with higher affinity for PDHK2 (e.g. compound 1).

TABLE 1
Effect of a range of compounds on binding of fluorescent binder
A to PDHK2 and PDHK4

| | PDHK2 Ki | | PDHK4 Ki | |
|---|---|---|---|---|
| Compound | nM ± S.E.M. | n | nM ± S.E.M. | n |
| 1 | 54 ± 6 | 14 | 482 ± 36 | 14 |
| 2 | 458 ± 52 | 6 | 739 ± 88 | 4 |
| 3 | 161 ± 23 | 5 | 306 ± 64 | 3 |
| 4 | 430 ± 95 | 3 | 538 ± 16 | 3 |
| 5 | 62 ± 5 | 4 | 707 ± 84 | 3 |
| 6 | 6.1 ± 3 | 4 | 51 ± 12 | 5 |
| 7 | 43 ± 8 | 3 | 189 ± 4 | 3 |
| 8 | 743 ± 132 | 3 | 862 ± 18 | 3 |
| 9 | 125 ± 20 | 5 | 152 ± 28 | 4 |
| 10 | 187 ± 25 | 4 | 202 ± 50 | 3 |
| 11 | 136 ± 16 | 5 | 574 ± 92 | 3 |
| 12 | 63 ± 18 | 4 | 339 ± 21 | 4 |
| 13 | 42 ± 8 | 5 | 338 ± 44 | 4 |
| 14 | 119 ± 43 | 2 | 993 ± 412 | 2 |
| 15 | 59 ± 3 | 3 | 661 ± 8 | 3 |

1. An assay method for determining binding of a test compound to PDHK at a given site comprising
determining (a) the degree of polarisation of the light emitted by a PDHK complex formed by contacting in solution a known concentration of PDHK with a known concentration of a fluorescent compound known to bind to PDHK at that site, and (b) the degree of polarisation of the light emitted by a PDHK complex formed by contacting in solution the test compound, the same concentration of PDHK and the same concentration of the fluorescent compound,
a lesser value of the degree of polarisation (b) than that of (a) being taken as indicative of binding of the test compound to that site.

2. An assay method as claimed in claim 1 wherein the degree of polarisation (b) is determined for each of a range of concentrations of the test compound, in each case contacted with a fixed concentration of PDHK and fluorescent compound, and the resultant values are compared to estimate the concentration of test compound which reduces the degree of polarisation (a) by 50%.

3. An assay method as claimed in claim 1 or claim 2 wherein the fluorescent compound contains the radical (I): 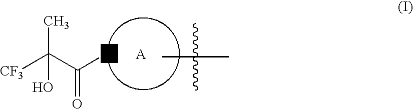
wherein ring A is mono or bicyclic:

4. An assay method as claimed in claim 1 wherein the fluorescent compound contains the radical (II)

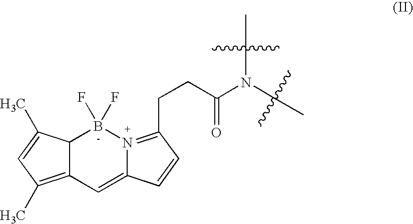

5. An assay method as claimed in claim 2 wherein the fluorescent compound contains the radical (II)

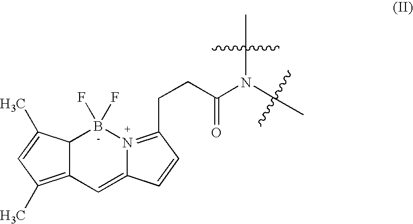

6. An assay method as claimed in claim 3 wherein the fluorescent compound contains the radical (II)

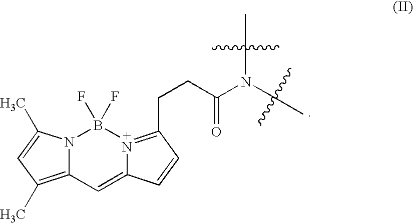

7. An assay method as claimed in claim 1 or claim 2 wherein the fluorescent compound has the structure (III)

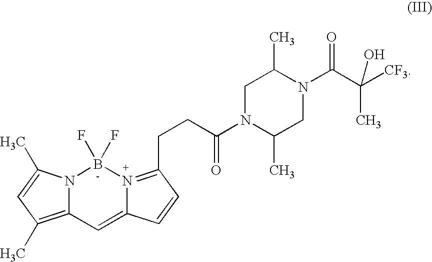

8. An assay method as claimed in claim 7 wherein the fluorescent compound has the stereoconfiguration shown in formula (IIIA):

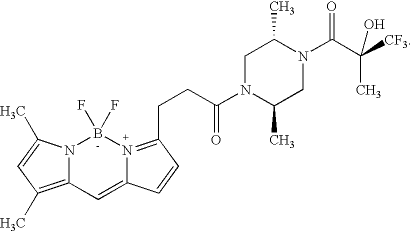

9. A method of screening a plurality of test compounds to identify any which bind to PDHK comprising subjecting the test compounds to an assay as claimed in any of the preceding claims.

10. A compound having the structure (III):

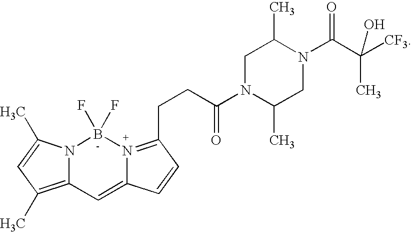

11. The compound of claim 10 having the structure (IIIA): [chemical structure]
